(19) United States
(12) Patent Application Publication
(10) Pub. No.: US 2014/0088015 A1
(43) Pub. Date: Mar. 27, 2014

(54) Mucin 3 EGF-like Domains

(71) Applicant: University of Minnesota, St. Paul, MN (US)

(72) Inventors: Samuel B. Ho, La Jolla, CA (US); Laurie L. Shekels, Mound, MN (US)

(21) Appl. No.: 13/871,312

(22) Filed: Apr. 26, 2013

Related U.S. Application Data
(63) Continuation of application No. 13/022,307, filed on Feb. 7, 2011, now abandoned.
Application No. 13/022,307 is a continuation of application No. 11/596,273, filed on Jul. 23, 2007, now abandoned.
(60) Provisional application No. 60/570,722, filed on May 13, 2004.

(51) Int. Cl. C07K 14/47

(52) U.S. Cl. CPC C07K 14/4727
USPC 514/13.2; 514/20.9; 514/20.8; 514/18.6; 530/395

(57) ABSTRACT

The invention provides for a mucin3 polypeptide, a polypeptide including a mucin3 EGF like domain, and nucleic acids encoding such polypeptides. The invention also provides for methods of treating an individual that has or is at risk of developing a disease or condition of the alimentary canal using such polypeptides or nucleic acids.

### PRIORITY CLAIM

This application is a continuation of U.S. patent application Ser. No. 13/022,307, filed Feb. 7, 2011, which is a continuation of U.S. patent application Ser. No. 11/596,273, filed Nov. 13, 2006, which is the U.S. National Phase of PCT Application No. PCT/US2005/016794 filed May 13, 2005, which claims the priority of U.S. Application No. 60/570,722, which was filed May 13, 2004. The aforementioned applications are incorporated herein in their entirety.

### FEDERALLY SPONSORED RESEARCH OR DEVELOPMENT

The U.S. Government may have certain rights in this invention pursuant to a Veterans Affairs Merit Review Award.

### TECHNICAL FIELD

This invention relates to epidermal growth factor (EGF) domains, and more particularly to EGF domains within mucin polypeptides.

### BACKGROUND

Mucins are a family of secreted and cell surface glycoproteins expressed by most epithelial tissues. Mucins are directed to the surface of epithelial tissues and are thought to play a protective role. Alterations in mucin proteins have been noted in conditions such as gastritis and peptic ulcer disease, Crohn's disease, ulcerative colitis, and intestinal cancers. Mucins can be grouped into two categories, secreted mucin proteins or membrane-bound mucin proteins. Secreted mucins are characterized by carboxyl and amino terminal domains termed “Von Willebrand-type D” domains that flank a large serine and threonine-rich domain that is heavily glycosylated. These mucins are able to join end-to-end to form long polymers that make them highly viscous in solution. Membrane-bound mucins are characterized by a carboxyl terminal domain containing a small cytoplasmic domain, a hydrophobic membrane-spanning domain, and an extracellular domain that is characterized in some cases by a cysteine-rich domain and a large serine and threonine rich glycosylated domain. Messenger RNA splice variants of these genes have been described that encode proteins without the membrane-spanning domain, which allows them to function as a secreted monomeric mucin. In this regard the membrane-spanning mucins can be considered bi-functional, existing as both membrane-associated proteins and as a secreted protein.

Many different proteins contain EGF-like domains, called G-modules. EGF-like domains are found in several growth factors as well as in numerous extracellular proteins involved in formation of the extracellular matrix, cell adhesion, chemotaxis, and wound healing. The six cysteines found in EGF-like domains form three intramolecular disulfide bonds creating a structural domain, which is important in maintaining protein-protein interactions or perhaps protein-membrane interactions. This domain or G-module consists of two small double-stranded beta sheets held together by disulfide bonds. Some but not all EGF-like domains are able to bind the EGF receptor.

### SUMMARY

In one aspect, the invention provides for an isolated nucleic acid that includes a nucleic acid molecule encoding a mucin3 EGF-like domain. Representative sequences include SEQ ID NOs: 3, 4, 5, 6, 9, 11, 12, and 14. The invention provides for constructs containing such nucleic acids. A construct can contain multiple mucin3 EGF-like domains (e.g., 2, 3, 4, 5, 6, or more). When multiple mucin3 EGF-like domains are present, the domains generally are separated by a linker region. Linker regions can be at least 100 amino acids in length. The sequences of representative linker regions are shown in SEQ ID NO:10 or 13. A mucin3 EGF-like domain can be a mouse mucin3 EGF-like domain or a human mucin3 EGF-like domain. Alternatively, mouse and human mucin3 EGF-like domains can be present together in a construct.

In another aspect, the invention provides methods of treating an individual that has or is at risk of developing a disease or condition of the alimentary canal. Such a method typically includes administering an effective amount of a polypeptide comprising a mucin3 EGF-like domain. Representative mucin3 EGF-like domains have the sequence shown in SEQ ID NOs: 3, 4, 5, 6, 9, 11, 12, and 14. Representative diseases of the alimentary canal include, without limitation, gastritis, peptic ulcer disease, Crohn's disease, ulcerative colitis, and intestinal cancers. Typically, an effective amount is an amount effective to stimulate cell migration or wound healing in the alimentary canal.

In another aspect, the invention provides for methods of treating or preventing an epithelial lesion in an individual. Such a method typically includes administering an effective amount of a polypeptide comprising a mucin3 EGF-like domain. Representative mucin3 EGF-like domains have the sequence shown in SEQ ID NOs: 3, 4, 5, 6, 9, 11, 12, and 14. Representative epithelial lesions include, for example, a lesion of the upper alimentary canal, the esophagus, the dermis, the epidermis, the vagina, the cervix, the uterus, the gastrointestinal tract, the distal bowel, the respiratory epithelium, and/or the corneal epithelium.

Mucin3 EGF-like domains generally do not directly activate an EGF receptor. In addition, mucin3 EGF-like domains can stimulate phosphorylation of proteins; usually proteins that are about 160 to about 200 kDa in size.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1. (A) Spacing of cysteines in the cysteine-rich region of mouse Muc3 and human MUC3 and MUC17. Cysteine spacing of EGF and trefoil motifs are shown for comparison. (B) Diagram of recombinant mouse GST-Muc3 fusion proteins expressed and purified from E. coli. Numbers correspond to the amino acids in the original Muc3 cDNA sequence described previously (Shekels et al., 1998, Biochem. J., 330:1301-1308).

FIG. 2. (A) Effect of recombinant GST peptide, m3EGF1,2 and recombinant EGF on A431 cell number after 24 hours, expressed as percent of control cell numbers in serum free medium. (B) Proliferation of Lovo colon cancer cells as measured by MTT after 24 hours. Negative control consisted of serum free media in Tris buffer and positive control was cells grown in 10% fetal bovine serum (FBS).

FIG. 3. Percent of total wound closure. Wounds were made in Young adult mouse colon (YAMC) cell monolayers and measured at 24 hours. EGF (1 ng/ml) was used as a positive control and resulted in 100% wound closure after 24 hours.

FIG. 4. (A) A431 cell migration in response to m3EGF1,2, m3EGF1, m3EGF2 over 18-24 hours represented as the percent of control cell number migrating in control serum free (SF) medium. (B) Migration of Lovo cells treated with varying concentrations of peptides represented as the percentage of control cells migrating in serum free medium after 24 hours. N=6 wells for each condition.

FIG. 5. (A) Mean number of cA431 cells migrating over 24 hours in response to m3EGF1,2 (10 μg/ml) or EGF (1 ng/ml) with and without the specific EGF/ErbB1 receptor inhibitor tyrphostin, AG1478 (150 nm). (B) Mean number of cA431 cells migrating over 24 hours in response to m3EGF1,2 (10 μg/ml) or EGF (1 ng/ml) with and without a general inhibitor of tyrosine phosphorylation, genistein (Gen, 15 μg/ml). SF=serum free medium, N=6 wells for each treatment.

FIG. 6. (A) YAMC cells were exposed to EGF (1 ng/ml) for 5 min or serum free media (SF), mEGF1,2 (10 μg/ml), or GST (10 μg/ml) for 30 min.

FIG. 7. (A) Percent change in apoptosis with (+) or without (−) TNF-α (100 ng/ml) treatment for 48 hrs. Cells lines included parental Lovo, LhM3c14, Lmock, and parental Lovo cells pretreated with m3EGF1,2 (10 μg/ml) or GST (5 μg/ml) for 1 hr prior to addition of TNF-α. (B) Percent change in apoptosis with (+) or without (−) sequential interferon gamma and anti-fas antibody treatment for 72 hours. Cell lines included LhM3c14 and Lmock.

FIG. 8. (A) Crypt damage score (CDS) at 30 hours post acetic acid administration in mice that received treatment with m3EGF1,2 (100 μg) or control peptide BSA (100 μg) in PBS per rectum at 12 and 24 hours following acetic acid. (B) Mean number of low power (10×) fields per specimen with complete grade III ulceration at 30 hours post acetic acid administration in mice treated with 100 μg m3EGF1,2 or control peptide 100 μg BSA in PBS. (C) Crypt damage score (CDS) at 30 hours post acetic acid administration in mice that received treatment with GST, m3EGF1 (EGF1), m3EGF2 (EGF2), or m3EGF1,2 per rectum at 12 and 24 hours following acetic acid. (D) Mean number of low power (10×) fields per specimen with complete grade III ulceration at 30 hours post acetic acid administration in mice that received treatment with GST, m3EGF1, m3EGF2, or m3EGF1,2 per rectum at 12 and 24 hours following acetic acid.

FIG. 9. Crypt damage scores and mean number of fields/specimen with grade III ulceration from the middle to distal mouse colons (A, B) and the proximal colons (C, D) are represented. Scores from control mice treated with GST and BSA were added together under “All Controls”.

FIG. 10. Nucleotide and amino acid sequences of human and mouse mucin3. Like reference symbols in the various drawings indicate like elements.

### DETAILED DESCRIPTION

The intestinal membrane-bound mucin gene, Muc3, encodes a large, membrane-bound mucin with an extracellular domain consisting of one large glycosylated tandom repeat domain and one domain with two cysteine-rich domains that have some similarity with epidermal growth factor (EGF)-like motifs or domains. Muc3 is highly expressed in the intestinal tract.

### Nucleic Acids

The present invention is based, in part, on the identification of Muc3 nucleic acid molecules and

EGF-like domains within Muc3 nucleic acid molecules. Nucleic acid molecules of the invention include, for example, the sequences shown in SEQ ID NO:17 or 19. Additional mucin3 nucleic acids can be found, for example, in GenBank Accession Nos. BC058768, AF450241, AF450242, and AF450243. As used herein, the term “nucleic acid molecule” can include DNA molecules and RNA molecules and analogs of the DNA or RNA molecule generated using nucleotide analogs. A nucleic acid molecule of the invention can be single-stranded or double-stranded, and the strandedness will depend upon its intended use.

The invention further encompasses nucleic acid molecules that differ from the nucleotide sequences shown in SEQ ID NO:17 or 19, or GenBank Accession Nos. BC058768, AF450241, AF450242, or AF450243. Nucleic acid molecules of the invention include molecules that are at least 10 nucleotides in length and that have at least 75% sequence identity (e.g., at least 80%, 85%, 90%, 95%, or 99% sequence identity) to any of the sequences shown in SEQ ID NO:17 or 19, or GenBank Accession Nos. BC058768, AF450241, AF450242, and AF450243. Nucleic acid molecules that differ in sequence from the nucleic acid sequences shown in SEQ ID NO:17 or 19, or GenBank Accession Nos. BC058768, AF450241, AF450242, and AF450243 can be generated by standard techniques, such as site-directed mutagenesis or PCR-mediated mutagenesis. In addition, nucleotide changes can be introduced randomly along all or part of a nucleic acid molecule encoding an EGF-like domain, such as by saturation mutagenesis. Alternatively, nucleotide changes can be introduced into a sequence by chemically synthesizing a nucleic acid molecule having such changes. Generally, human mucin genes and proteins are indicated in upper case letters, while mouse mucin genes and proteins are indicated in lower case letters.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It will be appreciated that a single sequence can align differently with other sequences and hence, can have different percent sequence identity values over each aligned region. It is noted that the percent identity value is usually rounded to the nearest integer. For example, 78.1%, 78.2%, 78.3%, and 78.4% are rounded down to 78%, while 78.5%, 78.6%, 78.7%, 78.8%, and 78.9% are rounded up to 79%. It is also noted that the length of the aligned region is always an integer.

The alignment of two or more sequences to determine percent sequence identity is performed using the algorithm described by Altschul et al. (1997, Nucleic Acids Res., 25:3389-3402) as incorporated into BLAST (basic local alignment search tool) programs, available at ncbi.nlm.nih.gov on the World Wide Web. BLAST searches can be performed to determine percent sequence identity between a nucleic acid molecule encoding a Muc3 EGF-like domain and any other sequence or portion thereof aligned using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a sequence of the invention and another sequence, the default parameters of the respective programs are used.

As used herein, an “isolated” nucleic acid molecule is a nucleic acid molecule that is separated from other nucleic acid molecules that are usually associated with the isolated nucleic acid molecule. Thus, an “isolated” nucleic acid molecule includes, without limitation, a nucleic acid molecule that is free of sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA, or genomic library) or a portion of a gel (e.g., agarose, or polyacrylamine) containing restriction-digested genomic DNA is not to be considered an isolated nucleic acid.

Isolated nucleic acid molecules of the invention can be obtained using techniques routine in the art. For example, isolated nucleic acids within the scope of the invention can be obtained using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid molecule of the invention. Isolated nucleic acids of the invention also can be chemically synthesized, either as a single nucleic acid molecule or as a series of oligonucleotides. In addition, isolated nucleic acid molecules of the invention also can be obtained by mutagenesis. For example, an isolated nucleic acid that shares identity with an art known sequence can be mutated using common molecular cloning techniques (e.g., site-directed mutagenesis). Possible mutations include, without limitation, deletions, insertions, substitutions, and combinations thereof.

A nucleic acid molecule also can contain multiple mucin3 EGF-like domains. For example, a nucleic acid molecule can contain two mucin3 EGF-like domains, three mucin3 EGF-like domains, four mucin3 EGF-like domains, or more. Typically, each mucin3 EGF-like domain is separated from another mucin3 EGF-like domain by a linker region. A linker region can include amino acids (e.g., from 5 to 150 amino acids), a chemical linkage, or a combination thereof.

Constructs containing nucleic acid molecules encoding one or more Muc3 EGF-like domains also are provided by the invention. Constructs, including expression vectors, suitable for use in the present invention are commercially available and/or produced by recombinant DNA technology methods routine in the art. A construct containing a Muc3 nucleic acid molecule can have elements necessary for expression operably linked to such a Muc3 nucleic acid, and further can include sequences such as those encoding a selectable marker (e.g., an antibiotic resistance gene), and/or those that can be used in purification of a polypeptide containing an EGF-like domain (e.g., 6×His tag).

Elements necessary for expression include nucleic acid sequences that direct and regulate expression of nucleic acid coding sequences. One example of an element necessary for expression is a promoter sequence. Elements necessary for expression also can include introns, enhancer sequences, response elements, or inducible elements that modulate expression of a nucleic acid. Elements necessary for expression can be of bacterial, yeast, insect, mammalian, or viral origin and vectors can contain a combination of elements from different origins. Elements necessary for expression are described, for example, in Goeddel, 1990, Gene Expression Technology: Methods in Enzymology, 185, Academic Press, San Diego, Calif. As used herein, operably linked means that a promoter and/or other regulatory element(s) are positioned in a vector relative to a nucleic acid in such a way as to direct or regulate expression of the nucleic acid. Many methods for introducing nucleic acids into cells, both in vivo and in vitro, are well known to those skilled in the art and include, without limitation, calcium phosphate precipitation, electroporation, heat shock, lipofection, microinjection, and viral-mediated nucleic acid transfer.

Another aspect of the invention pertains to host cells into which a vector of the invention, e.g., an expression vector, or an isolated nucleic acid molecule of the invention has been introduced. The term “host cell” refers not only to the particular cell but also to the progeny or potential progeny of such a cell. A host cell can be any prokaryotic or eukaryotic cell. For example, nucleic acids encoding Muc3 EGF-like domains can be expressed in bacterial cells such as E. coli, or in insect cells, yeast or mammalian cells (such as Chinese hamster ovary cells (CHO) or COS cells). Other suitable host cells are known to those skilled in the art.

Vectors containing Muc3 nucleic acid molecules were deposited with the American Type Culture Collection (ATCC), 10801 University Boulevard Manassas, Va. 20110. Each deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. §112.

### Polypeptides

One aspect of the invention pertains to purified mucin3 EGF-like domain polypeptides, as well as mucin3 EGF-like domain polypeptide fragments. Representative mucin3 EGF-like domains are shown in SEQ ID NOs:3, 4, 5, and 6, which each exhibit a unique cysteine pattern. The amino acid sequence of the first mouse mucin3 and the human MUCIN3 EGF-like domains are shown in SEQ ID NOs:12 and 9, respectively; the amino acid sequence of the mouse mucin3 and the human MUCIN3 linker region are shown in SEQ ID NOs:13 and 10, respectively; and the amino acid sequence of the second mouse mucin3 and the human MUCIN3 EGF-like domains are shown in SEQ ID NOs:14 and 11, respectively. The amino acid sequence of the human and mouse mucin3 are shown in SEQ ID NOs:18 and 20. The mucin17 EGF-like domains also are shown in SEQ ID NOs:7 and 8, and also demonstrate a unique cysteine pattern.

The term “purified” polypeptide as used herein refers to a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the proteins and naturally occurring molecules with which it is naturally associated. Since a polypeptide that is chemically synthesized is, by nature, separated from the components that naturally accompany it, a synthetic polypeptide is “purified.” Polypeptides can be purified from natural sources (e.g., a biological sample) by known methods such as DEAE ion exchange, gel filtration, and hydroxyapatite chromatography. A purified polypeptide also can be obtained by expressing a nucleic acid in an expression vector, for example. In addition, a purified polypeptide can be obtained by chemical synthesis. The extent of purity of a polypeptide can be measured using any appropriate method, e.g., column chromatography, polyacrylamide gel electrophoresis, or HPLC analysis.

In addition to naturally-occurring polypeptides, the skilled artisan will further appreciate that changes can be introduced into a nucleic acid molecule (e.g., those having the sequence shown in SEQ ID NO:17 or 19, or GenBank Accession Nos. BC058768, AF450241, AF450242, and AF450243) as discussed herein, thereby leading to changes in the amino acid sequence of the encoded polypeptide. For example, changes can be introduced into Muc3 nucleic acid coding sequences leading to conservative and/or non-conservative amino acid substitutions at one or more amino acid residues. A “conservative amino acid substitution” is one in which one amino acid residue is replaced with a different amino acid residue having a similar side chain. Similarity between amino acid residues has been assessed in the art. For example, Dayhoff et al. (1978, in Atlas of Protein Sequence and Structure, Vol. 5, Suppl. 3, pp 345-352) provides frequency tables for amino acid substitutions that can be employed as a measure of amino acid similarity. A non-conservative substitution is one in which an amino acid residue is replaced with an amino acid residue that does not have a similar side chain.

The invention also provides for chimeric or fusion polypeptides. As used herein, a “chimeric” or “fusion” polypeptide includes one or more Muc3 polypeptides operatively linked to a heterologous polypeptide. A heterologous polypeptide can be at either the N-terminus or C-terminus of the Muc3 polypeptide. Within a chimeric or fusion polypeptide, the term “operatively linked” is intended to indicate that the two polypeptides are encoded in-frame relative to one another. In a fusion polypeptide, the heterologous polypeptide generally has a desired property such as the ability to purify the fusion polypeptide (e.g., by affinity purification). A chimeric or fusion polypeptide of the invention can be produced by standard recombinant DNA techniques, and can use commercially available constructs.

A polypeptide commonly used in a fusion polypeptide for purification is glutathione S-transferase (GST), although numerous other polypeptides are available and can be used. In addition, a proteolytic cleavage site can be introduced at the junction between a Muc3 polypeptide and a non-Muc3 polypeptide to enable separation of the two polypeptides subsequent to purification of the fusion polypeptide. Enzymes that cleave such proteolytic sites include Factor Xa, thrombin, or enterokinase. Representative expression vectors encoding a heterologous polypeptide that can be used in affinity purification of a Muc3 polypeptide include pGEX (Pharmacia Biotech Inc; Smith & Johnson, 1988, Gene, 67:31-40), pMAL (New England Biolabs, Beverly, Mass.) and pRITS (Pharmacia, Piscataway, N.J.).

### Methods of Using Mucin3 EGF-Like Domains

The invention provides methods for preventing or treating a disease of the alimentary canal in an individual who has or is at risk of developing a disease of the alimentary canal. The invention also provides methods for treating an epithelial lesion in an individual. Individuals are treated by administering a polypeptide containing an EGF-like domain, or a nucleic acid encoding such a domain. Individuals at risk for a disease of the alimentary canal can be administered the polypeptide or nucleic acid prior to the manifestation of symptoms that are characteristic of a disease or condition of the alimentary canal, such that the disease or condition is prevented or delayed in its progression.

Diseases of the alimentary canal include, but are not limited to, gastritis, peptic ulcer disease, Crohn's disease, ulcerative colitis, or intestinal cancers. As used herein, epithelial lesion can refer to, without limitation, a lesion of the upper alimentary canal, the esophagus, the dermis, the epidermis, the vagina, the cervix, the uterus, the gastrointestinal tract, the distal bowel, the respiratory epithelium, or the corneal epithelium. Specifically, an epithelial lesion can be stomatitis, mucositits, gingivitis, a lesion caused by gastro-esophageal reflux disease, a traumatic lesion, a burn, a pressure ulcer, eczema, contact dermatitis, psoriasis, a herpetic lesion, acne, enteritis, proctitis, a lesion caused by Crohn's disease or ulcerative colitis, keratitis, a corneal ulcer, keratoconjunctivitis, a keratoconus, a conjunctiva, ocular inflammation, or a cicatricial pemphigoid. By way of example, a lesion as described herein can be caused by a bacterial, viral, protozoan, or fungal infection; by an allergic reaction, asthma, chronic obstructive pulmonary disease; by the inhalation of smoke, particulate matter, or a chemical; or by anti-neoplastic chemotherapy or anti-neoplastic radiation therapy.

In one embodiment, a compound administered to an individual can be a Muc3 polypeptide or a polypeptide containing a Muc3 EGF-like domain (e.g., Muc3EGF1 or Muc3EGF2; e.g., SEQ ID NOs: 3, 4, 5, 6, 9, 11, 12, or 14). A compound for administration can be a fusion polypeptide. In another embodiment, a compound administered to an individual can be a nucleic acid molecule encoding a Muc3 polypeptide or one or more Muc3 EGF-like domains. Nucleic acid coding sequences (e.g., full-length or otherwise) can be introduced into an appropriate expression vector such that a Muc3 or a Muc3 EGF-like domain or fusion polypeptide can be produced upon appropriate expression of the expression vector.

Compounds that can be used in compositions of the invention (e.g., nucleic acid molecules encoding a Muc3 polypeptide or a Muc3 EGF-like domain, or a Muc3 polypeptide or a polypeptide containing a Muc3 EGF-like domain) can be incorporated into pharmaceutical compositions suitable for administration. Such compositions typically comprise the nucleic acid molecule or polypeptide, and a pharmaceutically acceptable carrier. As used herein, “pharmaceutically acceptable carrier” is intended to include any and all solvents, dispersion media, coatings, antibacterial and anti-fungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., ingestion or inhalation), transdermal (topical), transmucosal, and rectal administration. Solutions or suspensions used for parenteral, intradermal, or subcutaneous application can include the following components: a sterile diluent such as water for injection, saline solution (e.g., phosphate buffered saline (PBS)), fixed oils, a polyol (for example, glycerol, propylene glycol, and liquid polyethylene glycol, and the like), glycerine, or other synthetic solvents; antibacterial and antifungal agents such as parabens, chlorobutanol, phenol, ascorbic acid, thimerosal, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol or sorbitol, and sodium chloride in the composition. Prolonged administration of the injectable compositions can be brought about by including an agent that delays absorption. Such agents include, for example, aluminum monostearate and gelatin. The parenteral preparation can be enclosed in ampoules, disposable syringes or multiple dose vials made of glass or plastic.

Oral compositions generally include an inert diluent or an edible carrier. Oral compositions can be liquid, or can be enclosed in gelatin capsules or compressed into tablets. Pharmaceutically compatible binding agents, and/or adjuvant materials can be included as part of an oral composition. Tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Transmucosal administration can be accomplished through the use of nasal sprays or suppositories. For transdermal administration, the active compounds are formulated into ointments, salves, gels, or creams as generally known in the art.

It is especially advantageous to formulate oral or parenteral compositions in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms of the invention are dependent upon the amount of a compound necessary to therapeutically treat the individual. The amount of a compound necessary can be formulated in a single dose, or can be formulated in multiple dosage units. Treatment of an individual may require a one-time dose, or may require repeated doses.

For therapeutic polypeptides, the dose typically is from about 0.1 mg/kg to about 100 mg/kg of body weight (generally, about 0.5 mg/kg to about 5 mg/kg). Modifications such as lipidation (Cruikshank et al., 1997, J. Acquired. Immune Deficiency Syndromes and Human Retrovirology, 14:193) can be used to stabilize polypeptides and to enhance uptake and tissue penetration. For nucleic acids, the dose administered will depend on the level of expression of the expression vector. Preferably, the amount of vector that produces an amount of a Muc3 polypeptide or a Muc3 EGF-like domain of from about 0.1 mg/kg to about 100 mg/kg of body weight is administered to an individual.

The invention will be further described in the following examples, which do not limit the scope of the invention described in the claims.

### EXAMPLES

### Example 1

### GST-Fusion Proteins

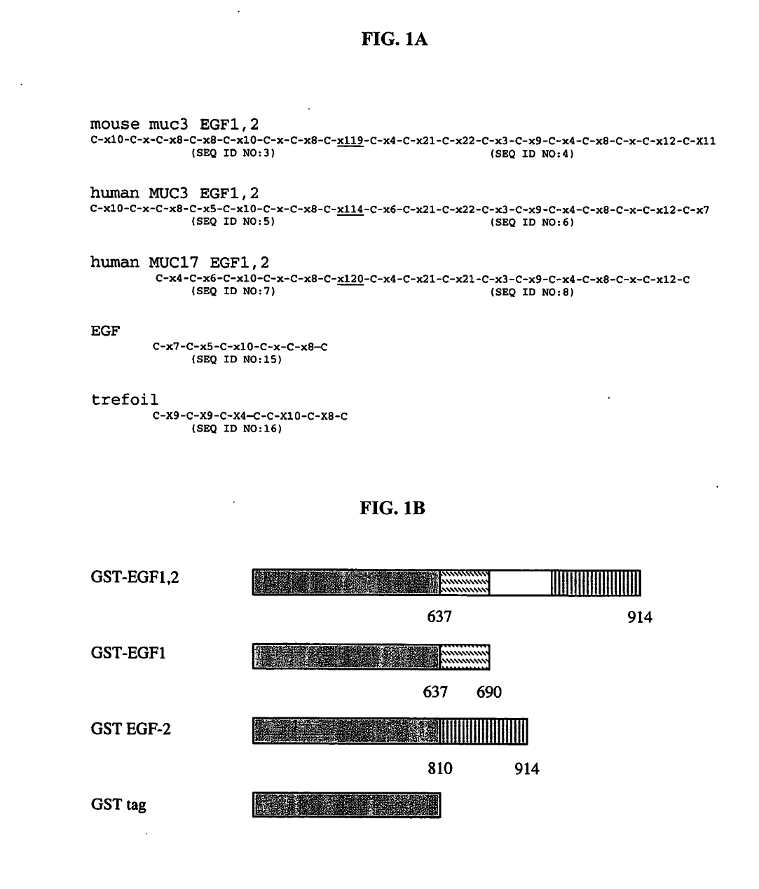

The extracellular region of mouse Muc3 including both EGF-like domains (m3EGF1,2) was amplified from mouse intestinal cDNA. In addition, products corresponding to only the first EGF-like domain (m3EGF1) or only the second EGF-like domain (m3EGF2) were also amplified. Amplification was performed as described previously (Shekels et al., 1998, Biochem. J., 330:1301-1308). The resulting fragments were cloned into the pGEX-2TK vector (Amersham, Piscataway, N.J.), sequenced, and introduced into E. coli strain BL21 (Invitrogen, Carlsbad, Calif.). GST-fusion proteins were then expressed in E. coli by induction with 0.5 mM IPTG (Fisher, Pittsburgh, Pa.) and purified by affinity chromatography using glutathione agarose (Sigma Chemical Co, St. Louis, Mo.). Fusion peptides containing both muc3 EGF-like domains (m3EGF1,2) or containing only the first EGF-like domain (m3EGF1) or only the second EGF-like domain (m3EGF2) were synthesized (FIG. 1C).

### Example 2

### Cell Culture

Mouse and human cells known to contain EGF-family receptors were used. A431 cells, an immortalized human epidermoid carcinoma cell line, were obtained from American Type Culture Collection (Manassas, Va.). A431 cells express high levels of EGF (ErbB1) receptor and migrate in response to EGF. Lovo cells are a human colon adenocarcinoma cell line and express ErbB1 and low level ErbB2 receptors. Lovo cells have previously been shown to express a truncated form of human MUC3 that lacks a portion of the EGF2 domain and the entire transmembrane domain.

Cells were grown in 24-well plates for cell migration and proliferation experiments or T-25 flasks for immunoblotting experiments using DMEM supplemented with 10% fetal calf serum+50 U penicillin/ml and 0.05 μg streptomycin/ml (Invitrogen, Carlsbad, Calif.). Cells were cultured at 37° C., 5% CO₂, 10% FCS until the desired confluence was reached. 24 hours before the experiments, the monolayers were washed with PBS and the cells were switched to serum-free media for cell migration and immunoblotting experiments or media containing 0.5% serum for cell proliferation experiments. Young adult mouse colon cells (YAMC) are conditionally immortalized mouse colon cells grown in RPMI 1640 supplemented with 5% FCS+50 U penicillin/ml and 0.05 μg streptomycin/ml.

### Example 3

### Cell Migration Assays

Confluent 24-well plates of A431 or Lovo cells were cultured overnight in serum-free medium, the medium was replaced with PBS, and the monolayers were mechanically wounded using a single edged razorblade as previously described (Burk et al., 1973, Proc. Nat. Acad. Sci. USA, 70:369-372). During inhibition experiments, cells were pre-incubated with 150 nM tyrphostin AG1478 (Sigma, St. Louis, Mo.) or 15 μg/ml genistein (Sigma, St. Louis, Mo.) for 30 min at 37° C. and then washed with PBS before wounding. After wounding, cells were rinsed twice with PBS and further incubated with the peptide of interest in DMEM for 18 to 24 h (37° C., 5% CO₂, 0% FCS). During inhibition experiments, cells were treated with the inhibitor and the peptide of interest for 18 h. After fixation and staining, those cells that had migrated from the wounded edge were counted at 100× using an inverted light microscope. Two successive fields were counted and averaged within one well, and three to twelve wells were averaged for each condition in each experiment. YAMC cells were grown to confluency, then a rotating disc was used to scrape cells from an area within a 24 well plate. After 20 hours the area of wound remaining was measured, as described previously (Frey et al., 2004, J. Biol. Chem., 279:44513-21).

### Example 4

### Cell Proliferation Assays

Cells were cultured in 24-well plates until they were at 60% confluency and then the cells were switched to media containing 0.5% serum for 24 h. After the monolayers were rinsed with PBS, they were incubated with the peptide of interest in DMEM for 24 h. Cells were quantitated by trypan blue staining (Kaiser et al., 1997, Gastroenterology, 112:1231-40). Two counts were averaged from each well; six wells were averaged per treatment. Proliferation for each treatment was represented as a percentage relative to the serum-free control. Cells also were grown in 96 well plates and cell numbers estimated by a tetrazolium-based colorimetric assay using dimethylthiazole diphenyltetrazolium bromide (MTT, Sigma, St. Louis, Mo.), as described previously (Shekels et al., 1995, J. Clin. Lab. Med., 127:57-66).

### Example 5

### Preparation of Cellular Lysates and Membranes

Cell monolayers were washed with PBS and then lysed in cell lysis buffer containing 0.5 M Tris pH 7.4, 0.25 M NaCl, 0.1% NP₄O, 0.05M EDTA, 2.9 M NaF. Cells were scraped from the flask and the lysate was incubated on ice for 10-15 min. After vortexing for 20 seconds, the lysate was centrifuged at 14,000 rpm for 10 min. Membranes were prepared from cells grown in T-75 flasks by the addition of a membrane lysis buffer containing 20 mM Tris HCl pH 8.0, 2 mM EDTA, 1 mM β-mercaptoethanol. Protease and phosphatase inhibitors were added prior to use. The monolayers were scraped into lysis buffer, put into ice-cold centrifuge tubes, and the monolayers were sheared using a 28-gauge needle. The lysate was centrifuged at 1000 rpm for 5 min and then the supernatant was centrifuged at 15,000 rpm for 30 minutes. The pellet containing the membranes was resuspended in 100 μl of RIPA lysis buffer and sheared using a 28-gauge needle. Reagents were purchased from Sigma, St. Louis, Mo.

### Example 6

### Immunoprecipitation and Immunoblotting

For immunoprecipitation, cell lysates or membrane preps were incubated with either anti-EGF receptor antibody, anti-ErbB2 receptor antibody, or anti-ErbB3 receptor antibody (all from Cell Signaling, Beverly, Mass.), at a 1:100 dilution overnight at 4° C.; after which Protein A beads (30 μl/300 μl lysate) were added for 2 hours. Immunoprecipitates were recovered by centrifugation and washed 3 times in lysis buffer. Pellets were resuspended in 2×SDS sample buffer and vortexed for 30 sec. Immunoprecipitates were denatured for 5 min at 100° C. and separated by SDS-PAGE before transfer to nitrocellulose membrane. After blocking for 2 h with 5% non-fat dried milk in TBS and washing 2×5 min with 0.05% Tween in TBS, Western blotting was conducted using an anti-phosphotyrosine monoclonal antibody (Cell Signaling) at a 1:2000 dilution overnight at 4° C. Control Western immunoblots were performed with the same samples using antibodies for the specific receptor that was immunoprecipitated at 1:2000 dilution overnight at 4° C. The membranes were washed twice with 0.05% Tween in TBS and then incubated for 1 hour with the peroxidase-conjugated secondary antibody (Sigma) at a 1:2000 dilution. After washing 4 times for 5 min each, proteins were visualized by chemiluminescence detection using Pierce Supersignal West Pico Chemiluminescent Substrate (Pierce Biotechnology, Rockford, Ill.). Immunoblotting was performed in a similar fashion on samples of cell lysates or membrane preps without prior immunoprecipitation, using anti-phosphotyrosine monoclonal antibody (Cell Signaling).

### Example 7

### Thiol Quantification in Recombinant Peptides

Determination of free cysteines in recombinant mucin proteins was performed using a method modified from Singh et al. (Singh et al., 1995, Methods Enzymol., 251:229-37). The Thiol and Sulfide Quantitation Kit from Molecular Probes (Eugene, Oreg.) was used. Briefly, recombinant mucin protein or control peptide was incubated with the inactive papain-SSCH₃. Free thiols in the protein reduce the papain-SSCH₃ to an active form. The activity of the reduced papain is measured using the chromogenic papain substrate, L-BAPNA (N-benzoyl-L-arginine, p-nitroanilide). Using the same method, a standard curve is prepared using a known concentration of L-cysteine. This standard curve is used to calculate the free thiol in the recombinant protein. A peptide corresponding to a tandem repeat sequence of the mouse Muc5AC (MGMtr) was used as a control peptide containing no cysteines (KQTSSPNTGKTSTISTT) (SEQ ID NO:1). EGF was also used as a control peptide. EGF has no free thiols, but 6 cysteines that are all involved in disulfide bonds. A peptide corresponding to a non-repetitive portion of the mouse Muc5AC (MGMnr) was used as a control peptide containing two free thiols (CKNELCNWTNWLDGSYPGSGRNSGD) (SEQ ID NO:2).

### Example 8

### Stable Transfection of Human MUC3 Cysteine-Rich Domain Construct

Primers corresponding to the human MUC3 EGF1,2 domain were synthesized and used to amplify human colon cDNA. The 936 bp human MUC3 EGF1,2 PCR product encoded the two human MUC3 EGF-like domains, the MUC3 transmembrane region, and 20 amino acids of the MUC3 cytoplasmic domain. The MUC3 PCR fragment was ligated to pFLAG-CMV-3 (Sigma). This vector encodes the preprotrypsin leader sequence, allowing for secretion of expressed proteins. The preprotrypsin leader sequence is followed by the FLAG tag at the amino terminus of the expressed protein of interest. The MUC3 transmembrane sequence targets the protein for insertion into the cell membrane. Confirmation of sequence and orientation of the insert was achieved by DNA sequencing.

Lovo cells were transfected with the human MUC3 transmembrane-EGF1,2 construct using Lipofectamine 2000 (Invitrogen). 48 hours after the start of transfection, cells were cultured in the presence of 800 μg/mL G418 (Invitrogen). G418-resistant clones were isolated using sterile cloning rings. Clone LhM3c14 was used for apoptosis assays. Lovo cells were also transfected with empty vector to generate a stable mock-transfected clone (Lmock). The transfectants were maintained in selective medium containing 800 μg/ml G418. Expression of the human MUC3 EGF1,2 construct was determined by Western blot analysis with rabbit anti-flag antibody (Sigma).

### Example 9

### Apoptosis Assays

Apoptosis was induced by adding 100 ng/ml TNF alpha (Sigma) to sub-confluent cultures of Lovo cells in 35 mm sterile Petri dishes in DMEM with 10% serum for 48 hours. Apoptosis was also induced by incubating cells with 1000 U/ml interferon gamma for 24 hours, followed by removal of the interferon and the addition of anti-fas antibody at 100-500 ng/ml for 72 hours (R&D Systems, Minneapolis, Minn.). Cells were fixed in 4% paraformaldehyde in (PBS pH 7.4) for 5 minutes, then washed twice in PBS. The cells were stained with the nuclear dye, Hoechst 33258 (Polysciences Inc., Warrington, Pa.), at a concentration of 5 μg/ml in PBS for 30 min, rinsed, cover-slipped with Slowfade Antifade (Molecular Probes, Eugene, Oreg.), and then immediately imaged using an ultraviolet microscope. Apoptotic nuclei were identified by morphology. The total number of normal and apoptotic nuclei were counted in three 40× lens fields per dish (representing >200 nuclei per dish). Three or more dishes were used for each experimental condition.

### Example 10

### Experimental Colitis Models

All experimental procedures were approved by the Institutional Animal Care and Use Committee at the Minneapolis Veterans Affairs Medical Center.

Acetic acid colitis: Female CD-1 mice (20-30 gm, Harlan Sprague Dawley, Indianapolis, Ind.) were fasted overnight and anesthetized with 3% isofluorane by inhalation. The rectum was then lavaged with 0.2 ml normal saline. Colitis was induced by intrarectal administration of 0.1 ml of 5% acetic acid. The solutions were administered through a trocar needle approximately 3 cm proximal to the anus. Mice were subsequently treated 12 and 24 hours later by intrarectal administration of 0.1 ml recombinant peptide in phosphate buffered saline or with 0.1 ml of control peptide in the same buffer at a similar concentration, using isofluorane anesthesia. All mice were harvested at 30 hours after induction of colitis (6-12 hours after the last treatment enema), and the distal colons were removed and examined for gross ulceration and microscopic examination. This model has been described previously (McCafferty et al., 1997, Gastroenterology, 112:1022-1027; and Tomita et al., 1995, Biochem J., 311:293-297).

Dextran Sodium Sulfate (DSS) colitis: Acute colitis was induced in female CD-1 mice (20-30 gm) by administration of 5% dextran sodium sulfate (molecular weight 40,000-50,000, USB, Cleveland, Ohio) in drinking water, as previously described (Okayasu et al., 1993, Gastroenterology, 98:694-702; Cooper et al., 1993, Lab. Invest., 69:238-49; Murthy et al., 1993, Dig. Dis. Sci., 38:1722-34). After 7 days, the DSS was removed from the drinking water. Mice were treated 24 and 48 hours after removal of DSS by intrarectal administration of 0.1 ml recombinant peptide in phosphate buffered saline or with 0.1 ml of control peptide in the same buffer, using isofluorane anesthesia. All mice were harvested at 72 hours after removal of DSS and the colons examined histologically.

### Example 11

### Histologic Mucosal Injury Score

Resected colons were fixed in 10% buffered formalin, embedded in paraffin, sectioned, and stained with hematoxylin and eosin. The severity of mucosal injury was graded similarly to that described previously (Okayasu et al., 1990, Gastroenterology, 98:694-702; Murthy et al., 1993, Dig. Dis. Sci., 38:1722-34). The injury scale was graded from 0 to III, as follows: grade 0=normal; grade I=distortion and/or destruction of the bottom third of glands and focal inflammatory infiltrate; grade II=erosions/destruction of all glands or the bottom two thirds of glands and inflammatory infiltrate with preserved surface epithelium; and grade III=loss of entire glands and surface epithelium. Specimens were examined without knowledge of the experimental group.

The total number of low power (10×) fields exhibiting grade III colitis was determined for each specimen. An overall crypt damage score was also calculated by giving grade I, II, and III scores of 1, 2, and 3, respectively. Each low power field was graded, and the percentage of each specimen with each score was calculated and added to give the final crypt damage score (range 0-3.00). For example, the same length of colon was examined for each specimen, and a specimen with 10% of fields with a score of 1, 25% of fields with a score of 2, and 25% of fields with a score of 3 would have a crypt damage score of (0.1)¹+(0.25)²+(0.25)³=1.35.

### Example 12

### Statistical Analysis

Mean±SEM was calculated for variables in each experimental group and analyzed using Student's t-test (two-tailed) and Fishers exact test. A p-value of <0.05 was considered significant.

### Example 13

### Design of Recombinant Muc3 Proteins

Recombinant GST fusion proteins corresponding to both mouse Muc3 EGF-like domains (m3EGF1,2), the first EGF-like domain (m3EGF1) or the second EGF-like domain (m3EGF2), were constructed, expressed in E. coli, and purified using glutathione-agarose columns. FIG. 1A shows the spacing of cysteines in the EGF-like domain of mouse Muc3 and human MUC3 and MUC17. Cysteine spacing of EGF and trefoil domains are shown for comparison. Note the highly conserved cysteine arrangement in the EGF-like domains of mouse Muc3 and human MUC3. The first and second EGF-like domains of Muc3 have 8 and 10 cysteines, respectively. The last 6 cysteines in each EGF-like domain are found in a spatial arrangement similar to EGF, with the second EGF-like domain showing less conservation of the spacing. No other significant sequence similarity is found between the Muc3 EGF-like domains and EGF.

Table 1 shows the cysteine arrangement and the amino acid sequence of the EGF 1 domain, the glycosylated linkage domain, and the EGF2 domain from mouse Muc3 and human MUC3. Human and mouse Muc3 share 60% and 44% overall sequence similarity between their first and second EGF-like domains. Comparison of the cysteine spacing of mouse Muc3 and human MUC17 shows less similarity, although the overall amino acid sequence similarity of mouse Muc3 and human MUC17 is comparable to the similarity with human MUC3 (52% and 64% sequence similarity in the first and second EGF-like domains, respectively).

TABLE 1
EGF-like Domains

| Mucin | EGF-like domain 1 | Linker region | EGF-like domain 2 |
|---|---|---|---|
| Mouse | C-x10-C-x-C-x8- | x119 | C-x4-C-x21-C-x22-C-x3-C- |
| MUC3 | C-x8-C-x10-C-x- | | x9-C-x4-C-x8-C-x-C-x12- |
| Cysteine | C-x8-C | | C-x11 |
| Spacing | (SEQ ID NO: 3) | | (SEQ ID NO:4) |
| Mouse | CMNGGFWTGD | EELVESVEIEPTVAASVGVS | CSALLCFNSTATKVQNS |
| MUC3 | KCICPNGFGGD | VTVTSQEYSEKLQDRKSEEF | ATVS VNPEETCKKEAGE |
| EGF1, 2 | RCENIVNVVNC | SNFNKTFTKQMALIYAGIPE | DFAKFVTLGQKGDKWF |
| | ENGGTWDGLK | YEGVIIKNLSKGSIVVDYDVI | CITPCSAGYSTSKNCSY |
| | CQCTSLFYGPR C | LKAKYTPGFENTLDTVVKN | GKCQLQRSGPQCLCLIT |
| | (SEQ ID NO: 12) | LETKIKNATEVQVQDVNNN | DTHWYSGENCDWGIQK |
| | | (SEQ ID NO: 13) | SLVYG |
| | | | (SEQ ID NO: 14) |
| Human | C-x10-C-x-C-x8- | x114 | C-x6-C-x21-C-x22-C-x3-C- |
| MUC3 | C-x5-C-x10-C-x- | | x9-C-x4-C-x8-C-x-C-x12-C- |
| Cysteine | C-x8-C | | x7 |
| Spacing | (SEQ ID NO: 5) | | (SEQ ID NO: 6) |
| Human | CDNGGTWEQG | EFAVEQVDLDVVETEVGME | CQDSQTLCFKPDSIKVN |
| MUC3 | QCACLPGFSGD | VSVDQQFSPDLNDNTSQAY | NNSKTELTPAAICRRAA |
| EGF1, 2 | RCQLQTRCQN | RDFNKTFWNQMQKIFADMQ | PTGYEEFYFPLVEATRL |
| | GGQWDGLKCQ | GFTFKGVEILSLRNGSIVVDY | RCVTKCTSGVDNAIDCH |
| | CPSTFYGSSC | LVLLEMPFSPQLESEYEQVK | QGQCVLETSGPTCRCYS |
| | (SEQ ID NO: 9) | TTLKEGLQNASQDVNS | TDTHWFSGPRCEVAVH |
| | | (SEQ ID NO: 10) | WR (SEQ ID NO: 11) |
| Human | C-x4-C-x6-C- | x120 | C-x4-C-x21-C-x21-C-x3-C- |
| MUC17 | x10-C-x-C-x8-C | | x9-C-x4-C-x8-C-x-C-x12-C |
| Cysteine | (SEQ ID NO: 7) | | (SEQ ID NO: 8) |
| Spacing | | | |

Rat Muc3 has been shown to be post-translationally cleaved at a SEA module and a second site lying between the two EGF-like domains. The resulting two subunits re-associate through a non-covalent bond that can be broken by 2% SDS and boiling. Recombinant m3EGF1,2 appeared as a predominant single band in reducing coomassie-stained gels at the expected molecular weight of 54 kDa. Treatment of recombinant m3EGF1,2 by boiling for 5 min in 2% SDS did not result in a change in molecular weight, indicating that this type of cleavage did not occur in the recombinant GST fusion protein. Similarly, the recombinant m3EGF1 and the m3EGF2 appeared as single bands of 34 kDa and 40 kDa, respectively, on reducing coomassie-stained gels.

To insure that disulfide bonds were formed in the recombinant mucin proteins, the free thiol content of the proteins was determined. The thiol content was determined to be near zero in control peptides (mouse gastric mucin tandem repeat peptide (MGMtr) and EGF) which are predicted to lack free thiols. The positive control peptide mouse gastric mucin non-repeat peptide MGMnr containing two free thiols was measured to contain 1.6 free cysteines per peptide (Table 2). GST alone also had negligible free thiols. m3EGF1,2 and m3EGF1 had very little measurable thiol, suggesting that all the cysteines were found in disulfide bonds. Interestingly, m3EGF2 appeared to have a free cysteine.

TABLE 2
Thiol measurement in recombinant peptides

| | Predicted # cysteines | Measured # free cysteines |
|---|---|---|
| Peptide | in sequence | per peptide |
| GST | 4 | 0.05 |
| GST-79 (m3EGF1,2) | 22 | 0.34 |
| GST-EGF1 (m3EGF1) | 12 | 0.12 |
| GST-EGF2 (m3EGF2) | 14 | 1.37 |
| EGF | 6 | 0.01 |
| MGMtr tandem repeat | 0 | 0.00 |
| MGMnr nonrepetitive | | |
| peptide | 2 | 1.57 |

### Example 14

### Effect of Muc3 Recombinant Peptides on Cell Proliferation

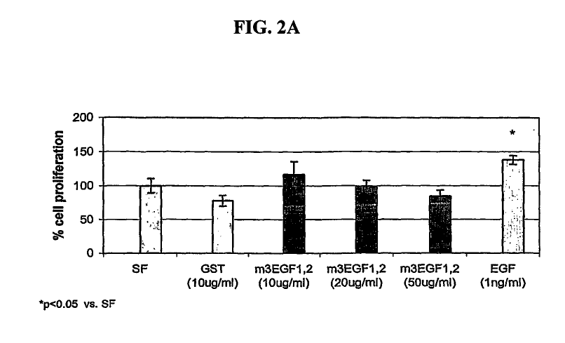

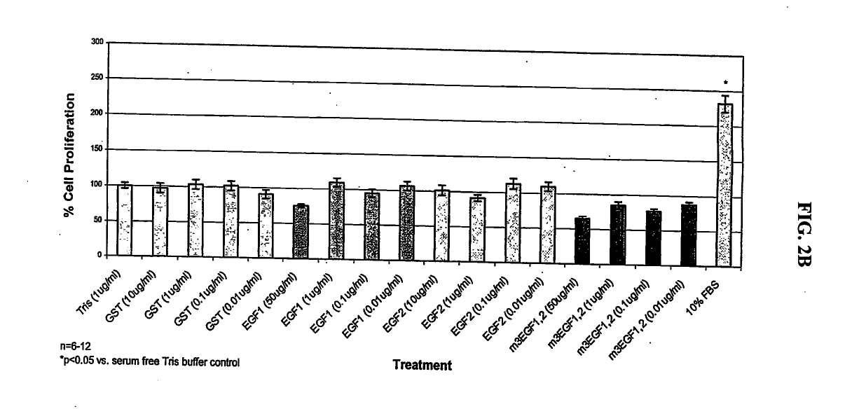

The effect of muc3 recombinant peptides on cell proliferation was determined in Lovo and A431 cells over 24 hours. As depicted in FIG. 2A, treatment of Lovo cells with m3EGF1, m3EGF2, m3EGF1,2 did not result in any significant changes in cell numbers after 24 hours. Similarly, there is no significant effect on cell numbers after treatment of YAMC and A431 cells with 10

### 50 μg/ml of m3EGF1,2 (FIG. 2B). No effect on cell proliferation was observed in YAMC cells treated with 10-50 μg/ml of m3EGF1,2.

### Example 15

### Recombinant m3EGF1.2 Stimulates Cell Migration

Mouse colonic cells (YAMC), human epithelial cell lines A431, and Lovo human colon cancer cells, known to contain ErbB receptors, were examined to determine if recombinant Muc3 EGF domain proteins stimulated cell migration.

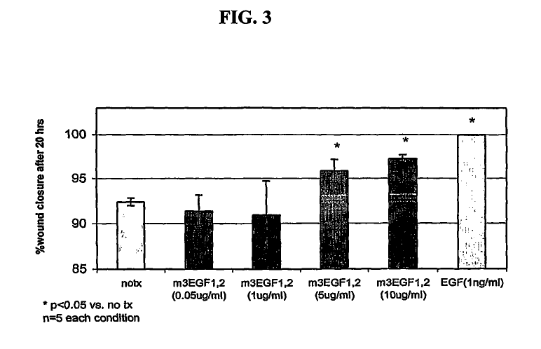

YAMC cells treated with m3EGF1,2 demonstrated significantly increased wound closure over 20 hours compared with control treatment (p<0.05), and a dose response was demonstrated (FIG. 3). Human A431 cells treated with 10 μg/ml m3EGF1,2 for 18-24 hours demonstrated a 215% increase in cell migration above controls (p<0.05).

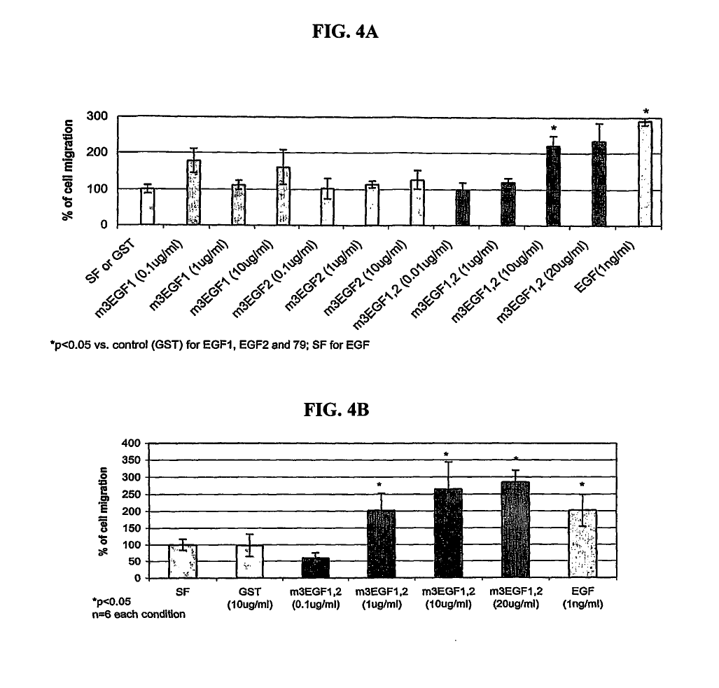

In A431 cells, recombinant EGF at 1 μg/ml stimulated cell migration to nearly 300% of controls. In contrast, the truncated Muc3 cysteine rich recombinant proteins m3EGF1 and m3EGF2 did not alter cell migration (FIG. 4A).

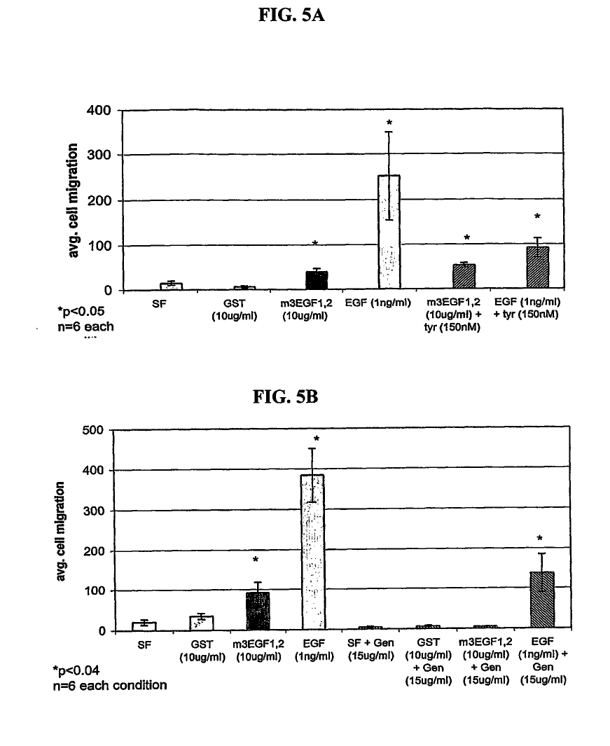

Lovo human colon cancer cells treated with 1 μg/ml of m3EGF1,2 demonstrated a 2 fold increase in cell migration over 24 hours compared with controls, which was similar to the migration induced by 1 ng/ml recombinant EGF (FIG. 4B). A dose response was demonstrated with a further 2.6-fold increase in cell migration with 10 μg/ml of m3EGF1,2. Subsequent increases in cell migration with doses of 20 μg/ml or more were not observed. In order to determine if recombinant Muc3 EGF domain proteins acted via stimulation of the EGF receptor, an inhibitor of this receptor, AG1478, was used to pre-treat A431 cells. The inhibitor, at 150 nm of AG1478, inhibited EGF-induced cell migration, but not cell migration induced by m3EGF1,2 (FIG. 5A). To determine if tyrosine phosphorylation was required for m3EGF1,2-induced cell migration, A431 cells were pre-treated with 15 μg/ml genistein. This resulted in significant inhibition of EGF-induced cell migration and complete inhibition of cell migration induced by m3EGF1,2 (FIG. 5B).

### Example 16

### Recombinant m3EGF1,2 does not Activate EGF Receptors

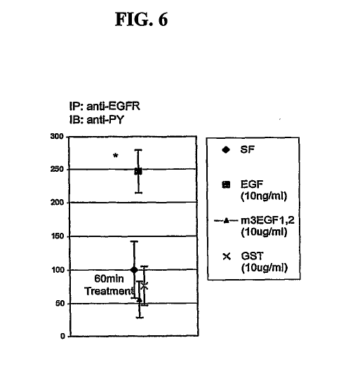

To further analyze whether m3EGF1,2 caused activation or phosphorylation of the EGF (ErbB1) receptor, A431 cells were treated with recombinant proteins and cell lysates were examined for overall phosphotyrosine content. The EGF receptor was immunoprecipitated and analyzed by immunoblot using an anti-phosphotyrosine antibody to assess EGF receptor phosphorylation. Treatment of cells with recombinant EGF at 1 ng/ml for 1, 30 and 60 minutes resulted in a significant increase in a 175 kD band of phosphotyrosine content compared with control treatments. In contrast, no change in 175 kD phosphotyrosine reactivity in 175 kD bands was observed in A431 cells treated with m3EGF1,2 or control GST peptide at 1, 30, and 60 minutes. This was confirmed by EGF (ErbB1) receptor immunoprecipitation followed by phosphotyrosine blotting. Triplicate experiments demonstrated a significant increase in EGF receptor phosphorylation by recombinant EGF, but not by m3EGF1,2 or control peptide at 60 minutes (FIG. 6A). Subconfluent cultures of YAMC cells were similarly treated with 10 μg/ml of m3EGF1,2 and a similar concentration of GST for 30 minutes, or with 1 ng/ml recombinant EGF for 5 minutes. Cell lysates were immunoprecipitated with antibodies to EGF receptor, ErbB2, and ErbB3. Phosphorylation of EGFr and ErbB2 occurred in response to EGF, however m3EGF1,2 treatment did not result in phosphorylation of EGFr, ErbB2, or ErbB3 (FIG. 6A).

### Example 17

### Endogenous MUC3 and Exogenous Muc3 Peptides Inhibit Apoptosis

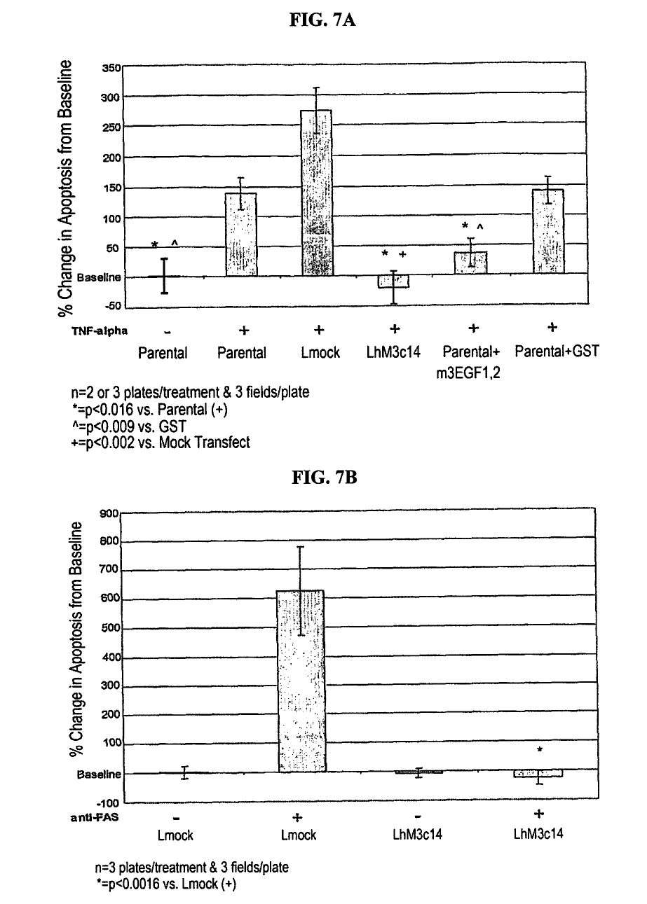

A human MUC3A transmembrane-EGF1,2 domain construct was stably transfected into Lovo human colon cancer cells. Lovo cell clone LhM3c14 expressed high levels of flag-tagged human MUC3A EGF1,2 in the cell membrane fractions; this was absent from LhM3c14 cytoplasmic fractions, mock transfected Lovo cells (Lmock) and parental Lovo cells. Apoptosis was induced in parental Lovo human colon cells and Lmock cells using TNF-alpha. The stable transfectant clone LhM3c14 was markedly resistant to TNF-alpha induced apoptosis (FIG. 7A). Similarly, pretreatment of parental Lovo cells with 100 μg/m1 m3EGF1,2 reduced TNF alpha-induced apoptosis, whereas pre-treatment with control GST peptide did not (FIG. 7B). Apoptosis induced by sequential interferon gamma and anti-fas antibody treatment was markedly reduced in the stable transfectant clone LhM3c14 compared to the mock transfectant Lmock (FIG. 7B).

### Example 18

### Recombinant m3EGF1,2 Accelerates Healing of Experimental Colitis

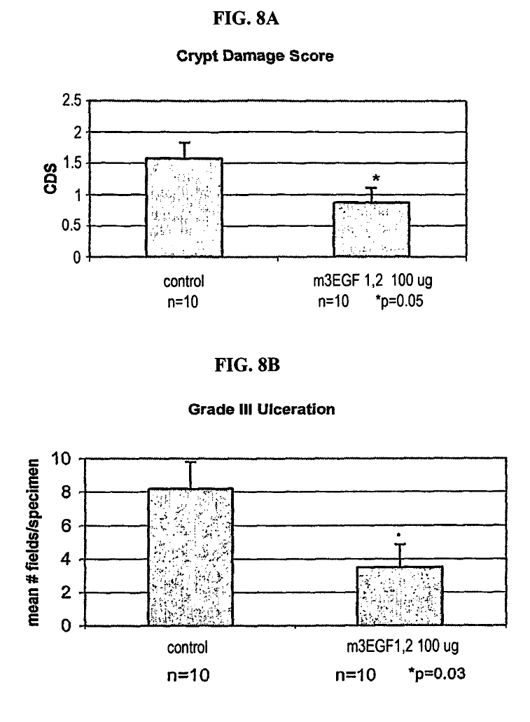

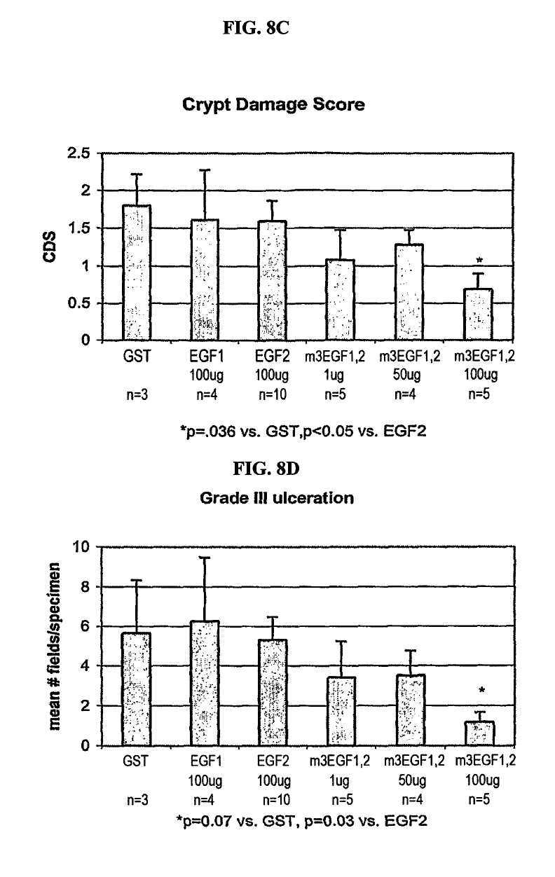

To determine if recombinant peptides could influence the healing or regeneration of intestinal mucosa, two different mouse models of acute colitis were used. In the first model, acute colonic injury was induced in mice by 5% acetic acid enemas, followed by the administration of recombinant protein or control enemas at 12 and 24 hours. The animals were sacrificed at 30 hours to determine the extent of mucosal damage. Treatment of mice with 100 μg m3EGF1,2 per rectum at 12 and 24 hours following acetic acid reduced total crypt damage score by 45% compared with enemas containing 100 μg BSA in PBS buffer (p=0.05) (FIG. 8A). This was largely due to the significant reduction in total or grade III mucosal ulceration from 8.2±1.6 low power fields/specimen in control treated mice to 3.5±1.4 low power fields/specimen in mice treated with 100 μm3EGF1,2 peptide enemas (p=0.038) (FIG. 8B).

Histologic differences were observed between normal mouse colonic mucosa and grade I, grade II, and grade Ill damage. The experiment was repeated using control enemas containing PBS buffer with 100 μg of recombinant GST, compared with enemas containing 1 μg, 50 μg, or 100 μg of recombinant m3EGF1,2; 100 μm3EGF1; and 100 μm3EGF2. Mice treated at 12 and 24 hours with enemas containing 100 μg of m3EGF1,2 demonstrated a significant 62% reduction in crypt damage score (FIG. 8C) and a 79% reduction in grade 111 mucosal ulceration (FIG. 8D) compared with mice treated with enemas containing 100 μg GST control protein. Mice treated with enemas containing 1 μm3EGF1,2 and 50 μm3EGF1,2 had non-significant reductions of 29-40% in crypt damage scores and 38-40% in grade III ulceration compared with control enema treatment. In contrast, enemas containing 100 μg m3EGF1 or 100 μm3EGF2 had no effect on crypt damage score or total mucosal ulceration (FIG. 8C,D).

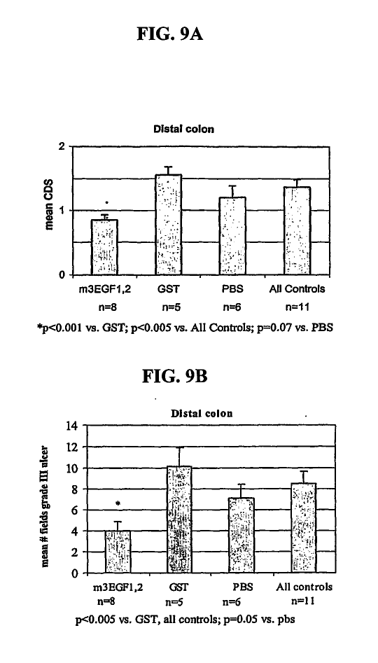

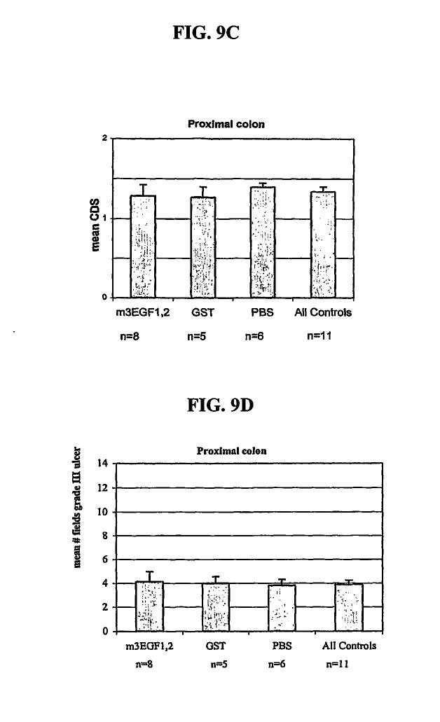

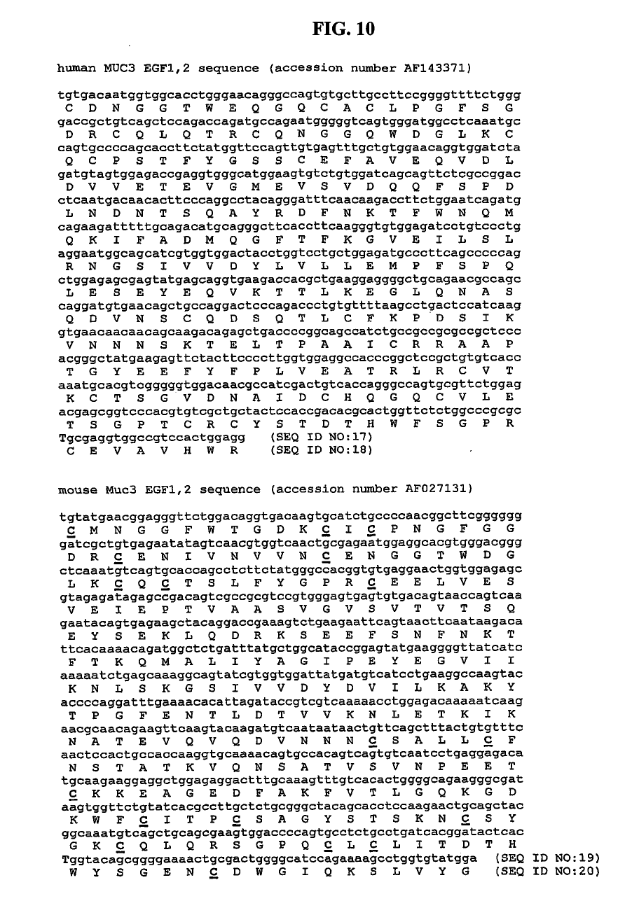

Administration of 5% DSS in drinking water for 7 days results in an acute colitis that predominates in the distal colon and heals with withdrawal of the DSS. Mice treated with 100 μg m3EGF1,2 per rectum at 12 and 24 hours after DSS withdrawal and examined at 72 hours after DSS withdrawal demonstrated a 38% reduction in crypt damage scores in the distal colon compared with mice treated with control enemas with GST or BSA (p<0.005) (FIG. 9A). This was primarily due to a 53% decrease in the mean number of fields/specimen with total grade III mucosal ulceration; from a mean of 8.5±1.1 fields/specimen in all controls to 4.0±0.8 fields/specimen in mice treated with m3EGF1,2 (p<0.005) (FIG. 9B). Mucosal damage was less in the proximal colon, and no significant differences were observed in crypt damage scores or in the number of fields with grade III ulceration in treated and control mice (FIG. 9C,D).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1-31. (canceled)

32. A method of treating an individual that has or is at risk of developing a disease or condition of the alimentary canal, comprising: administering an effective amount of a polypeptide comprising a mucin17 EGF-like domain.

33. The method of claim 32, wherein said mucin17 EGF-like domain comprises a sequence shown in SEQ ID NOs:7 or 8.

34. The method of claim 32, wherein said disease or condition of the alimentary canal is selected from the group consisting of gastritis, peptic ulcer disease, Crohn's disease, ulcerative colitis, and intestinal cancers.

35. The method of claim 32, wherein said effective amount is an amount effective to stimulate cell migration or wound healing in the alimentary canal.

36. A method of treating or preventing an epithelial lesion in an individual, comprising: administering an effective amount of a polypeptide comprising a mucin17 EGF-like domain.

37. The method of claim 36, wherein said polypeptide comprising a mucin17 EGF-like domain has a sequence shown in SEQ ID NOs:7 or 8.

38. The method of claim 36, wherein said epithelial lesion is a lesion of the upper alimentary canal, the esophagus, the dermis, the epidermis, the vagina, the cervix, the uterus, the gastrointestinal tract, the distal bowel, the respiratory epithelium, or the corneal epithelium.

39. The method of claim 36, wherein said epithelial lesion is stomatitis, mucositits, gingivitis, a lesion caused by gastro-esophageal reflux disease, a traumatic lesion, a burn, a pressure ulcer, eczema, contact dermatitis, psoriasis, a herpetic lesion, acne, enteritis, proctitis, a lesion caused by Crohn's disease or ulcerative colitis, keratitis, a corneal ulcer, keratoconjunctivitis, a keratoconus, a conjunctiva, ocular inflammation, or a cicatricial pemphigoid.

40. The method of claim 32, wherein said polypeptide comprising a mucin17 EGF-like domain comprises two or more mucin17 EGF-like domains.

41. The method of claim 40, wherein each of said two or more mucin17 EGF-like domains is separated from the adjacent of said two or more mucin17 EGF-like domains by a linker region, wherein each linker region independently comprises from 5 to 150 amino acids, a chemical linkage or a combination thereof.

42. The method of claim 36, wherein said polypeptide comprising a mucin17 EGF-like domain comprises two or more mucin17 EGF-like domains.

43. The method of claim 42, wherein each of said two or more mucin17 EGF-like domains is separated from the adjacent of said two or more mucin17 EGF-like domains by a linker region, wherein each linker region independently comprises from 5 to 150 amino acids, a chemical linkage or a combination thereof.

44-47. (canceled)

48. A purified polypeptide consisting essentially of a polypeptide selected from the group consisting of human mucin17 EGF1, mucin17 EGF2, mouse and human MUC17 EGF1,2.

49. A pharmaceutical composition comprising an effective amount of a polypeptide comprising a mucin17 EGF-like domain and a pharmaceutically acceptable carrier.

50. A method of treating an individual that has or is at risk of developing a disease or condition of the alimentary canal, comprising: administering an effective amount of a polypeptide comprising human mucin17 EGF1, human mucin17 EGF2, or human MUC17 EGF1,2.

51. A method of treating or preventing an epithelial lesion in an individual, comprising: administering an effective amount of a polypeptide comprising human mucin17 EGF1, or human MUC17 EGF1,2.

52-55. (canceled)

56. The purified polypeptide of claim 48, wherein said polypeptide consists of a mucin17 EGF-like domain consisting of a sequence shown in SEQ ID NO: 7.

57. The purified polypeptide of claim 48, wherein said polypeptide consists of a mucin17 EGF-like domain consisting of a sequence shown in SEQ ID NO: 8.

58. The purified polypeptide of claim 48, wherein said polypeptide consists of a mucin17 EGF-like domain consisting of two or more mucin17 EGF-like domains.

59. The purified polypeptide of claim 58, wherein each of said two or more mucin17 EGF-like domains is separated from the adjacent of said two or more mucin17 EGF-like domains by a linker region, wherein each linker region independently consists of 5 to 150 amino acids, a chemical linkage or a combination thereof.

60. (canceled)

61. The pharmaceutical composition of claim 49, wherein said polypeptide consists of a mucin17 EGF-like domain consisting of a sequence shown in SEQ ID NO: 7.

62. The pharmaceutical composition of claim 49, wherein said polypeptide consists of a mucin17 EGF-like domain consisting of a sequence shown in SEQ ID NO: 8.

63. The pharmaceutical composition of claim 49, wherein said polypeptide consists of a mucin17 EGF-like domain consisting of two or more mucin17 EGF-like domains.

64. The pharmaceutical composition of claim 63, wherein each of said two or more mucin17 EGF-like domains is separated from the adjacent of said two or more mucin17 EGF-like domains by a linker region, wherein each linker region independently consists of 5 to 150 amino acids, a chemical linkage or a combination thereof.


SEQUENCE LISTING (20 sequences)

SEQ ID NO: 1 - protein, 17 aa, Artificial Sequence
  Synthetic peptide
KQTSSPNTGKTSTISTT

SEQ ID NO: 2 - protein, 25 aa, Artificial Sequence
  Synthetic peptide
CKNELCNWTNWLDGSYPGSGRNSGD

SEQ ID NO: 3 - protein, 54 aa, Mus musculus
  DOMAIN (1)..(54): Muc3 EGF-like domain 1 - Xaa equals any of the naturally occurring amino acids
CXXXXXXXXXXCXCXXXXXXXXCXXXXXXXXCXXXXXXXXXXCXCXXXXXXXXC

SEQ ID NO: 4 - protein, 105 aa, Mus musculus
  DOMAIN (1)..(105): Mouse Muc3 EGF-Like domain 2 - Xaa equals any of the naturally occurring amino acids
CXXXXCXXXXXXXXXXXXXXXXXXXXXCXXXXXXXXXXXXXXXXXXXXXXCXXXCXXXXXXXXXCXXXXCXXXXXXXXCXCXXXXXXXXXXXXCXXXXXXXXXXX

SEQ ID NO: 5 - protein, 51 aa, Homo sapiens
  DOMAIN (1)..(51): Human Muc3 EGF-like domain 1 - Xaa equals any of the naturally occurring amino acids
CXXXXXXXXXXCXCXXXXXXXXCXXXXXCXXXXXXXXXXCXCXXXXXXXXC

SEQ ID NO: 6 - protein, 103 aa, Homo sapiens
  DOMAIN (1)..(103): Human Muc3 EGF-Like Domain 2 - Xaa equals any of the naturally occurring amino acids
CXXXXXXCXXXXXXXXXXXXXXXXXXXXXCXXXXXXXXXXXXXXXXXXXXXXCXXXCXXXXXXXXXCXXXXCXXXXXXXXCXCXXXXXXXXXXXXCXXXXXXX

SEQ ID NO: 7 - protein, 35 aa, Homo sapiens
  DOMAIN (1)..(35): Human Muc17 EGF-Like Domain 1 - Xaa equals any of the naturally occurring amino acids
CXXXXCXXXXXXCXXXXXXXXXXCXCXXXXXXXXC

SEQ ID NO: 8 - protein, 93 aa, Homo sapiens
  DOMAIN (1)..(93): Human EGF-Like Domain 2 - Xaa equals any of the naturally occurring amino acids
CXXXXCXXXXXXXXXXXXXXXXXXXXXCXXXXXXXXXXXXXXXXXXXXXCXXXCXXXXXXXXXCXXXXCXXXXXXXXCXCXXXXXXXXXXXXC

SEQ ID NO: 9 - protein, 51 aa, Homo sapiens
  DOMAIN (1)..(51): Human Muc3 EGF-Like Domain 1
CDNGGTWEQGQCACLPGFSGDRCQLQTRCQNGGQWDGLKCQCPSTFYGSSC

SEQ ID NO: 10 - protein, 114 aa, Homo sapiens
  MISC_FEATURE (1)..(114): Human Muc3 linker region
EFAVEQVDLDVVETEVGMEVSVDQQFSPDLNDNTSQAYRDFNKTFWNQMQKIFADMQGFTFKGVEILSLRNGSIVVDYLVLLEMPFSPQLESEYEQVKTTLKEGLQNASQDVNS

SEQ ID NO: 11 - protein, 103 aa, Homo sapiens
  DOMAIN (1)..(103): Human Muc3 EGF-Like Domain 2
CQDSQTLCFKPDSIKVNNNSKTELTPAAICRRAAPTGYEEFYFPLVEATRLRCVTKCTSGVDNAIDCHQGQCVLETSGPTCRCYSTDTHWFSGPRCEVAVHWR

SEQ ID NO: 12 - protein, 54 aa, Mus musculus
  DOMAIN (1)..(54): Mouse Muc3 EGF-Like Domain 1
CMNGGFWTGDKCICPNGFGGDRCENIVNVVNCENGGTWDGLKCQCTSLFYGPRC

SEQ ID NO: 13 - protein, 119 aa, Mus musculus
  MISC_FEATURE (1)..(119): Mouse Muc3 Linker Region
EELVESVEIEPTVAASVGVSVTVTSQEYSEKLQDRKSEEFSNFNKTFTKQMALIYAGIPEYEGVIIKNLSKGSIVVDYDVILKAKYTPGFENTLDTVVKNLETKIKNATEVQVQDVNNN

SEQ ID NO: 14 - protein, 105 aa, Mus musculus
  DOMAIN (1)..(105): Mouse Muc3 EGF-Like Domain 2
CSALLCFNSTATKVQNSATVSVNPEETCKKEAGEDFAKFVTLGQKGDKWFCITPCSAGYSTSKNCSYGKCQLQRSGPQCLCLITDTHWYSGENCDWGIQKSLVYG

SEQ ID NO: 15 - protein, 38 aa, Homo sapiens
  DOMAIN (1)..(38): EGF Domain - Xaa equals any of the naturally occurring amino acids
CXXXXXXXCXXXXXCXXXXXXXXXXCXCXXXXXXXXXC

SEQ ID NO: 16 - protein, 47 aa, Homo sapiens
  DOMAIN (1)..(47): Trefoil Domain - Xaa equals any of the naturally occurring amino acids
CXXXXXXXXXCXXXXXXXXXCXXXXCCXXXXXXXXXXCXXXXXXXXC

SEQ ID NO: 17 - DNA, 804 bp, Homo sapiens
tgtgacaatggtggcacctgggaacagggccagtgtgcttgccttccggggttttctggggaccgctgtcagctccagaccagatgccagaatgggggtcagtgggatggcctcaaatgccagtgccccagcaccttctatggttccagttgtgagtttgctgtggaacaggtggatctagatgtagtggagaccgaggtgggcatggaagtgtctgtggatcagcagttctcgccggacctcaatgacaacacttcccaggcctacagggatttcaacaagaccttctggaatcagatgcagaagatttttgcagacatgcagggcttcaccttcaagggtgtggagatcctgtccctgaggaatggcagcatcgtggtggactacctggtcctgctggagatgcccttcagcccccagctggagagcgagtatgagcaggtgaagaccacgctgaaggaggggctgcagaacgccagccaggatgtgaacagctgccaggactcccagaccctgtgttttaagcctgactccatcaaggtgaacaacaacagcaagacagagctgaccccggcagccatctgccgccgcgccgctcccacgggctatgaagagttctacttccccttggtggaggccacccggctccgctgtgtcaccaaatgcacgtcgggggtggacaacgccatcgactgtcaccagggccagtgcgttctggagacgagcggtcccacgtgtcgctgctactccaccgacacgcactggttctctggcccgcgctgcgaggtggccgtccactggagg

SEQ ID NO: 18 - protein, 268 aa, Homo sapiens
CDNGGTWEQGQCACLPGFSGDRCQLQTRCQNGGQWDGLKCQCPSTFYGSSCEFAVEQVDLDVVETEVGMEVSVDQQFSPDLNDNTSQAYRDFNKTFWNQMQKIFADMQGFTFKGVEILSLRNGSIVVDYLVLLEMPFSPQLESEYEQVKTTLKEGLQNASQDVNSCQDSQTLCFKPDSIKVNNNSKTELTPAAICRRAAPTGYEEFYFPLVEATRLRCVTKCTSGVDNAIDCHQGQCVLETSGPTCRCYSTDTHWFSGPRCEVAVHWR

SEQ ID NO: 19 - DNA, 834 bp, Mus musculus
tgtatgaacggagggttctggacaggtgacaagtgcatctgccccaacggcttcgggggggatcgctgtgagaatatagtcaacgtggtcaactgcgagaatggaggcacgtgggacgggctcaaatgtcagtgcaccagcctcttctatgggccacggtgtgaggaactggtggagagcgtagagatagagccgacagtcgccgcgtccgtgggagtgagtgtgacagtaaccagtcaagaatacagtgagaagctacaggaccgaaagtctgaagaattcagtaacttcaataagacattcacaaaacagatggctctgatttatgctggcataccggagtatgaaggggttatcatcaaaaatctgagcaaaggcagtatcgtggtggattatgatgtcatcctgaaggccaagtacaccccaggatttgaaaacacattagataccgtcgtcaaaaacctggagacaaaaatcaagaacgcaacagaagttcaagtacaagatgtcaataataactgttcagctttactgtgtttcaactccactgccaccaaggtgcaaaacagtgccacagtcagtgtcaatcctgaggagacatgcaagaaggaggctggagaggactttgcaaagtttgtcacactggggcagaagggcgataagtggttctgtatcacgccttgctctgcgggctacagcacctccaagaactgcagctacggcaaatgtcagctgcagcgaagtggaccccagtgcctctgcctgatcacggatactcactggtacagcggggaaaactgcgactggggcatccagaaaagcctggtgtatgga

SEQ ID NO: 20 - protein, 278 aa, Mus musculus
CMNGGFWTGDKCICPNGFGGDRCENIVNVVNCENGGTWDGLKCQCTSLFYGPRCEELVESVEIEPTVAASVGVSVTVTSQEYSEKLQDRKSEEFSNFNKTFTKQMALIYAGIPEYEGVIIKNLSKGSIVVDYDVILKAKYTPGFENTLDTVVKNLETKIKNATEVQVQDVNNNCSALLCFNSTATKVQNSATVSVNPEETCKKEAGEDFAKFVTLGQKGDKWFCITPCSAGYSTSKNCSYGKCQLQRSGPQCLCLITDTHWYSGENCDWGIQKSLVYG